(19) United States
(12) Patent Application Publication
(10) Pub. No.: US 2020/0197480 A1
(43) Pub. Date: Jun. 25, 2020

(54) COMBINATION COMPRISING A PARTICULAR POLYMYXIN

(71) Applicant: HELPERBY THERAPEUTICS LIMITED, London (GB)

(72) Inventors: Anthony COATES, London (GB); Yanmin HU, London (GB)

(21) Appl. No.: 16/620,743

(22) PCT Filed: Jun. 8, 2018
(86) PCT No.: PCT/GB2018/051569

§ 371 Date: Dec. 9, 2019

(30) Foreign Application Priority Data
Jun. 9, 2017 (GB) 1709193.5

(51) Int. Cl. A61K 38/12; C07D 401/06; A61P 31/04

(52) U.S. Cl. CPC A61K 38/12; A61P 31/04; C07D 401/06

(57) ABSTRACT

The invention provides a combination comprising a compound of formula (I) or a compound of formula (II) or pharmaceutically acceptable derivatives or prodrugs thereof and a polymyxin selected from polymyxin E and polymyxin B or a pharmaceutically acceptable derivative thereof. This combination is particularly useful for the treatment of microbial infections.

### CROSS-REFERENCE TO RELATED APPLICATION

This application is a 35 U.S.C. § 371 national stage filing of PCT Application No. PCT/GB2018/051569 filed on Jun. 8, 2018, which claims priority to Great Britain Patent Application No. 1709193.5 filed on Jun. 9, 2017, each of which are incorporated herein in their entirety by reference.

### FIELD OF THE INVENTION

This invention relates to a combination comprising a compound of formula (I) or a compound of formula (II) defined herein or a pharmaceutically acceptable derivative or prodrug thereof, and a polymyxin selected from polymyxin E and polymyxin B, or a pharmaceutically acceptable derivative thereof. Such a combination is particularly useful for the treatment of microbial infections.

### BACKGROUND OF THE INVENTION

Before the introduction of antibiotics, patients suffering from acute microbial infections (e.g. tuberculosis or pneumonia) had a low chance of survival. For example, mortality from tuberculosis was around 50%. Although the introduction of antimicrobial agents in the 1940s and 1950s rapidly changed this picture, bacteria have responded by progressively gaining resistance to commonly used antibiotics. Now, every country in the world has antibiotic-resistant bacteria.

Indeed, more than 70% of bacteria that give rise to hospital acquired infections in the USA resist at least one of the main antimicrobial agents that are typically used to fight infection (Nature Reviews, Drug Discovery 1, 895-910 (2002)). In its 2014 report of global antimicrobial resistance, the World Health Organization focussed on the high levels of antibiotic resistance in the bacteria that cause common infections.

The global problem of advancing antimicrobial resistance has also led to a renewed interest in polymyxins. Polymyxins are a group of closely related antibiotic substances with a general structure consisting of a cyclic peptide and a hydrophobic tail. They are produced by non-ribosomal peptide synthetase systems in Gram-positive bacteria such as Paenibacillus polymyxa, and are selectively toxic for Gram-negative bacteria due to their specificity for the lipopolysaccharide molecule that exists within many Gram-negative outer membranes. Polymyxins B and E are used in the treatment of Gram-negative bacterial infections.

There have, however, been reports which show that even polymyxin E (colistin) may be losing its effectiveness in antibacterial therapy. The U.S. Military HIV Research Program has for instance reported colistin resistance in a human E. coli infection (www.sciencedaily.com/releases/2016/05/160526152033.htm).

In order to develop more long-term solutions to the problem of bacterial resistance, it is clear that alternative approaches are required. One such approach is to co-administer another drug or compound with the failing antibiotic so as to restore sufficient antibacterial activity. These compounds are often called “antibiotic resistance breakers” (ARBs), and their use to restore antibiotics is exemplified by the successful co-administration of β-lactamase inhibitors, such as clavulanic acid, with β-lactam antibiotics, such as amoxicillin (White, A. R. et al., J. Antimicrob. Chemother. 53 (Suppl. 1), i3-i20 (2004); Prabhudesai, P. P. et al., J. Indian Med. Assoc. 109, 124-127 (2011)).

The Applicant's International Patent Application published as WO2012032360 discloses a combination comprising phenoxybenzamine or a pharmaceutically acceptable derivative thereof and a polymyxin selected from polymyxin E and polymyxin B or a pharmaceutically acceptable derivative thereof, and its use in treating a microbial infection.

WO2014147405 then discloses the use of colistin (polymyxin E) in combination with zidovudine for treating a microbial infection. WO2016097754 discloses a combination comprising suloctidil or a pharmaceutically acceptable derivative or prodrug thereof, and a polymyxin selected from polymyxin E and polymyxin B or a pharmaceutically acceptable derivative thereof, and its use in treating a microbial infection.

Bacterial resistance to these combinations is, however, inevitable. Consequently there is an ongoing need in the art for new combinations which have activity against bacterial infections, particularly Gram-negative bacterial infections. There is also an urgent need for combinations which are effective against multi drug-resistant Gram-negative bacteria.

This need is met with the present invention because a compound of formula (I) or a compound of formula (II) defined herein or pharmaceutically acceptable derivatives or prodrugs thereof was surprisingly discovered to be an effective antibiotic resistance breaker when combined with a polymyxin or pharmaceutically acceptable derivative thereof.

The inventors surprisingly found that this combination exhibited synergistic antibacterial activity against bacteria, particularly against Gram-negative bacteria. In other words, the combination was unexpectedly found to have a greater biological activity than the expected additive effect of each agent at the stated dosage level. The surprising biological activity of the combination of the present invention offers the opportunity to shorten chemotherapy regimens and may result in a reduction in the emergence of microbial resistance associated with the use of such combination.

Advantageously, and as described below, the combination of the present invention has also been demonstrated to be particularly effective against drug-resistant bacteria, particularly drug-resistant Gram-negative bacteria. This opens the way for the combination to be administered both to drug-resistant strains and in said strains before drug-resistance is built up, i.e. as a first line treatment.

Synergy in the context of antimicrobials drugs is measured in a number of ways that conform to the generally accepted opinion that “synergy” is an effect greater than additive. One of the ways to assess whether synergy has been observed is to use the “chequerboard” technique. This is a well-accepted method that leads to the generation of a value called the fractional inhibitory concentration index (FICI).

Orhan et al J. Clin. Microbiol. 2005, 43(1):140 for instance describe the chequerboard method and analysis in the paragraph bridging pages 140-141. This document explains that the FICI value is a ratio of the sum of the MIC (Minimum Inhibitory Concentration) level of each individual component alone and in the mixture. The combination is considered synergistic when the ΣFIC is ≤0.5, indifferent when the ΣFIC is >0.5 to <2, and antagonistic when the ΣFIC is ≥2.

Another accepted test for ascertaining the presence or absence of synergy is to use time-kill methods where the dynamic effect of a drug combination is compared to each drug alone when assessing the effect on bacterial log or stationary-growth over time. Again, the possible results are synergy, indifference, or antagonism.

### SUMMARY OF THE INVENTION

Thus, in one embodiment the present invention provides a combination comprising a compound of formula (I) or a pharmaceutically acceptable derivative or prodrug thereof and a polymyxin selected from polymyxin E and polymyxin B, or a pharmaceutically acceptable derivative thereof; wherein the compound has the formula:

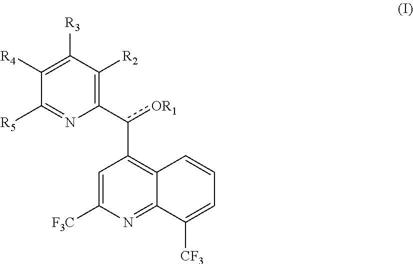

wherein R₁ is H, alkyl, alkenyl or COR^{a}, wherein R^{a} is hydrogen, alkyl, alkenyl, alkynyl, aryl, heteroaryl, carbocyclyl, heterocyclyl, or alkoxy; or R₁ is absent and a double bond is present;
wherein R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl, aryl, alkenyl, cycloalkyl, cycloalkenyl, heteroaryl, carbocyclyl, heterocyclyl, or alkoxy; or
R₂ and R₃, R₃ and R₄ or R₄ and R₅ may together define a cycloalkyl, cycloalkenyl, aryl, heteroaryl or heterocyclyl group;
wherein R₂, R₃, R₄ and R₅ may be the same or different; and
wherein the alkyl, aryl, alkenyl, alkynyl, cycloalkyl, cycloalkenyl, heteroaryl, carbocyclyl, and heterocyclyl groups are optionally substituted.

In another embodiment the present invention provides a combination comprising a compound of formula (II) or a pharmaceutically acceptable derivative or prodrug thereof and a polymyxin selected from polymyxin E and polymyxin B, or a pharmaceutically acceptable derivative thereof; wherein the compound has the formula:

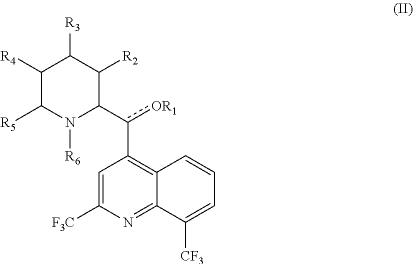

wherein R₁, R₂, R₃, R₄ and R₅ are as defined above for formula (I); and
wherein R₆ is selected from hydrogen, alkyl, aryl, alkenyl, alkynyl, cycloalkyl, cycloalkenyl, heteroaryl, carbocyclyl, or heterocycyl, wherein the alkyl, aryl, alkenyl, alkynyl, cycloalkyl, cycloalkenyl, heteroaryl, carbocyclyl, and heterocyclyl groups are optionally substituted.

Preferably R₆ is hydrogen or an alkyl group. More preferably R₆ is hydrogen.

The definitions of R₁, R₂, R₃, R₄ and R₅ set out herein are applicable to the compound of formula (I) and the compound of formula (II). Preferably R₁ is hydrogen. Alternatively R₁ is absent and a double bond is present. In the compound of formula (II), R₁ is preferably absent and a double bond is present.

Preferably R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl, alkenyl, or alkoxy. R₁ may also be hydrogen or absent. More preferably R₂, R₃, R₄ and R₅ are each independently hydrogen, hydroxy, halo, haloalkyl, haloalkoxy, alkyl, aryl, alkenyl, or alkoxy. Particularly preferred is where R₂, R₃, R₄ and R₅ are hydrogen.

For example, R₁, R₂, R₃, R₄ and R₅ may each independently be hydrogen so that the compound of formula (I) has the following chemical structure:

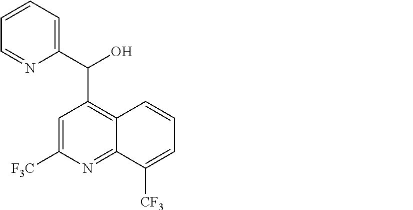 [SMILES: OC(c1ccccn1)c1cc(C(F)(F)F)nc2c(C(F)(F)F)cccc12]

This compound is referred to herein as HT0160010. Its chemical name is (2,8-bis-trifluoromethyl-quinolin-4-yl)-pyridin-2-yl-methanol.

Alternatively R₁ may be absent and R₂, R₃, R₄ and R₅ may each independently be hydrogen so that the compound of formula (I) has the following chemical structure:

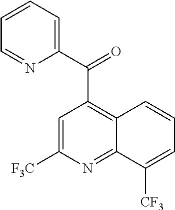 [SMILES: O=C(c1ccccn1)c1cc(C(F)(F)F)nc2c(C(F)(F)F)cccc12]

The chemical name for this compound is (2,8-bis-trifluoromethyl-quinolin-4-yl)-pyridin-2-yl-methanone.

When R₁, R₂, R₃, R₄, R₅ and R₆ are each independently hydrogen in the compound of formula (II), the compound has the following chemical structure:

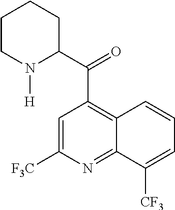 [SMILES: O=C(c1cc(C(F)(F)F)nc2c(C(F)(F)F)cccc12)C1CCCCN1]

This compound is referred to herein as HT0160009.

Preferably the polymyxin in the combination is polymyxin E or a pharmaceutically acceptable derivative thereof.

In a further embodiment the present invention provides the combination described herein for use in the treatment of a microbial infection. Preferably the combination is for use in killing multiplying, non-multiplying or clinically latent microorganisms associated with a microbial infection.

In a further embodiment the present invention provides the use of the combination described herein for the manufacture of a medicament for the treatment of a microbial infection.

In a further embodiment, the invention provides a method of treating a microbial infection which comprises administering the combination described herein to a subject (e.g. a human subject) in need thereof.

Preferably the infection is a bacterial infection. More preferably the infection is a Gram-negative bacterial infection. For example, the infection may be caused by Enterobacteriaceae, Klebsiella, Proteus, Acinetobacter, or Pseudomonas aeruginosa. Particularly preferred is an infection caused by Enterobacteriaceae or Klebsiella, e.g. an infection caused by E. coli or K.pneumoniae. Most preferred is an infection caused by E. coli.

There is also provided a pharmaceutical composition comprising a compound of formula (I) or a compound of formula (II) as described herein or pharmaceutically acceptable derivatives or prodrugs thereof in combination with a polymyxin selected from polymyxin E and polymyxin B or a pharmaceutically acceptable derivative thereof, and a pharmaceutically acceptable adjuvant, diluent or carrier. Preferably the pharmaceutical composition is for use in the treatment of a microbial infection.

In a further embodiment the invention provides a product comprising a compound of formula (I) or a compound of formula (II) as described herein or pharmaceutically acceptable derivatives or prodrugs thereof, and a polymyxin selected from polymyxin E and polymyxin B or a pharmaceutically acceptable derivative thereof as a combined preparation for simultaneous, separate or sequential use in the treatment of a microbial infection.

### DETAILED DESCRIPTION OF THE INVENTION

The afore-mentioned combination is useful for the treatment of a microbial infection. In particular, the afore-mentioned combination may be used to kill multiplying (log phase), non-multiplying (stationary phase) and/or clinically latent (persistent) microorganisms associated with microbial infections. References herein to the treatment of a microbial infection therefore include killing multiplying, non-multiplying and/or clinically latent microorganisms associated with such infections. In a preferred embodiment, the aforementioned combinations are used to kill non-multiplying and/or clinically latent microorganisms, most preferably non-multiplying microorganisms.

As used herein, the terms “combination” and “in combination with” refer to both separate and sequential administration of the compound of formula (I) or the compound of formula (II) and the polymyxin. When the agents are administered sequentially, either the compound or the polymyxin (e.g. polymyxin E) may be administered first. When administration is simultaneous, the agents may be administered either in the same or a different pharmaceutical composition. Adjunctive therapy, i.e. where one agent is used as the primary treatment and the other agent is used to assist that primary treatment, is also an embodiment of the present invention.

As used herein, “kill” means a loss of viability as assessed by a lack of metabolic activity.

As used herein, “clinically latent microorganism” means a microorganism that is metabolically active but has a growth rate that is below the threshold of infectious disease expression. The threshold of infectious disease expression refers to the growth rate threshold below which symptoms of infectious disease in a host are absent.

The metabolic activity of clinically latent microorganisms can be determined by several methods known to those skilled in the art; for example, by measuring mRNA levels in the microorganisms or by determining their rate of uridine uptake. In this respect, clinically latent microorganisms, when compared to microorganisms under logarithmic growth conditions (in vitro or in vivo), possess reduced but still significant levels of:
(I) mRNA (e.g. from 0.0001 to 50%, such as from 1 to 30, 5 to 25 or 10 to 20%, of the level of mRNA); and/or
(II) uridine (e.g. [³H]uridine) uptake (e.g. from 0.0005 to 50%, such as from 1 to 40, 15 to 35 or 20 to 30% of the level of [³H]uridine uptake).

Clinically latent microorganisms typically possess a number of identifiable characteristics. For example, they may be viable but non-culturable; i.e. they cannot typically be detected by standard culture techniques, but are detectable and quantifiable by techniques such as broth dilution counting, microscopy, or molecular techniques such as polymerase chain reaction. In addition, clinically latent microorganisms are phenotypically tolerant, and as such are sensitive (in log phase) to the biostatic effects of conventional antimicrobial agents (i.e. microorganisms for which the minimum inhibitory concentration (MIC) of a conventional antimicrobial is substantially unchanged); but possess drastically decreased susceptibility to drug-induced killing (e.g. microorganisms for which, with any given conventional antimicrobial agent, the ratio of minimum microbiocidal concentration (e.g. minimum bactericidal concentration, MBC) to MIC is 10 or more).

As used herein, the term “microorganisms” means fungi and bacteria. References herein to “microbial”, “antimicrobial” and “antimicrobially” shall be interpreted accordingly. For example, the term “microbial” means fungal or bacterial, and “microbial infection” means any fungal or bacterial infection. Preferably the microbial infection treated with the combination of the present invention is a bacterial infection. Particularly a Gram-negative bacterial infection, e.g. an infection caused by Enterobacteriaceae.

As used herein, the term “bacteria” (and derivatives thereof such as “bacterial infection”) includes, but is not limited to, references to organisms (or infections due to organisms) of the following classes and specific types:

Gram-positive cocci, such as:

Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus); Streptococci (e.g. beta-haemolytic, pyogenic streptococci (such as Strept. agalactiae, Strept. canis, Strept dysgalactiae dysgalactiae, Strept dysgalactiae equisimilis, Strept. equi equi, Strept equi zooepidemicus, Strept. iniae, Strept porcinus and Strept pyogenes), microaerophilic, pyogenic streptococci (Streptococcus “milleri”, such as Strept. anginosus, Strept constellatus constellatus, Strept constellatus pharyngidis and Strept. intermedius), oral streptococci of the “mitis” (alpha-haemolytic—Streptococcus “viridans”, such as Strept. mitis, Strept. oralis, Strept. sanguinis, Strept. cristatus, Strept gordonii and Strept. parasanguinis), “salivarius” (non-haemolytic, such as Strept salivarius and Strept. vestibularis) and “mutans” (tooth-surface streptococci, such as Strept. criceti, Strept. mutans, Strept ratti and Strept sobrinus) groups, Strept. acidominimus, Strept. bovis, Strept. faecalis, Strept. equinus, Strept pneumoniae and Strept suis, or Streptococci alternatively classified as Group A, B, C, D, E, G, L, P, U or V Streptococcus);

Gram-negative cocci, such as:

Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri; Bacillaceae, such as Bacillus anthracis, Bacillus subtilis, Bacillus thuringiensis, Bacillus stearothermophilus and Bacillus cereus; Enterobacteriaceae, such as Escherichia coli, Enterobacter (e.g. Enterobacter aerogenes, Enterobacter agglomerans and Enterobacter cloacae), Citrobacter (such as Citrob. freundii and Citrob. divernis), Hafnia (e.g. Hafnia alvei), Erwinia (e.g. Erwinia persicinus), Morganella morganii, Salmonella (Salmonella enterica and Salmonella typhi), Shigella (e.g. Shigella dysenteriae, Shigella flexneri, Shigella boydii and Shigella sonnei), Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis), Proteus (e.g. Pr. mirabilis, Pr. rettgeri and Pr. vulgaris), Providencia (e.g. Providencia alcalifaciens, Providencia rettgeri and Providencia stuartii), Serratia (e.g. Serratia marcescens and Serratia liquifaciens), and Yersinia (e.g. Yersinia enterocolitica, Yersinia pestis and Yersinia pseudotuberculosis); Enterococci (e.g. Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gaffinarum, Enterococcus hirae, Enterococcus malodoratus, Enterococcus mundtii, Enterococcus pseudoavium, Enterococcus raffinosus and Enterococcus solitarius); Helicobacter (e.g. Helicobacter pylori, Helicobacter cinaedi and Helicobacter fennelliae); Acinetobacter (e.g. A. baumanfi, A. calcoaceticus, A. haemolyticus, A. johnsonfi, A. junii, A. Iwoffi and A. radioresistens); Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri); Bacteroides fragilis; Peptococcus (e.g. Peptococcus niger); Peptostreptococcus; Clostridium (e.g. C. perfringens, C. difficile, C. botulinum, C. tetani, C. absonum, C. argentinense, C. baratii, C. bifermentans, C. beijerinckii, C. butyricum, C. cadaveris, C. camis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium); Mycoplasma (e.g. M. pneumoniae, M. hominis, M. genitalium and M. urealyticum); Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium lentiflavum, Mycobacterium malmoense, Mycobacterium mucogenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi); Haemophilus (e.g. Haemophilus influenzae, Haemophilus ducreyi, Haemophilus aegyptius, Haemophilus parainfluenzae, Haemophilus haemolyticus and Haemophilus parahaemolyticus); Actinobacillus (e.g. Actinobacillus actinomycetemcomitans, Actinobacillus equuli, Actinobacillus hominis, Actinobacillus lignieresii, Actinobacillus suis and Actinobacillus ureae); Actinomyces (e.g. Actinomyces israelii); Brucella (e.g. Brucella abortus, Brucella canis, Brucella melintensis and Brucella suis); Campylobacter (e.g. Campylobacter jejuni, Campylobacter coli, Campylobacter Ian and Campylobacter fetus); Listeria monocytogenes; Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus); Erysipelothrix rhusopathiae; Corynebacteriaceae (e.g. Corynebacterium diphtheriae, Corynebacterium jeikeum and Corynebacterium urealyticum); Spirochaetaceae, such as Borrelia (e.g. Borrelia recurrentis, Borrelia burgdorferi, Borrelia afzelii, Borrelia andersonfi, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. pertenue and Treponema carateum); Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis); Bordetella (e.g. Bordetella bronchiseptica, Bordetella hinzii, Bordetella holmseii, Bordetella parapertussis, Bordetella pertussis and Bordetella trematum); Nocardiaceae, such as Nocardia (e.g. Nocardia asteroides and Nocardia brasiliensis); Rickettsia (e.g. Ricksettsii or Coxiella burnetii); Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii); Moraxella catarrhalis; Cyclospora cayetanensis; Entamoeba histolytica; Giardia lamblia; Trichomonas vaginalis; Toxoplasma gondii; Stenotrophomonas maltophilia; Burkholderia cepacia; Burkholderia mallei and Burkholderia pseudomallei; Francisella tularensis; Gardnerella (e.g. Gardneralla vaginalis and Gardneralla mobiluncus); Streptobacillus moniliformis; Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena); Bartonella (Bartonella bacilliformis, Bartonella clarridgeiae, Bartonella elizabethae, Bartonella henselae, Bartonella quintana and Bartonella vinsonii arupensis); Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii); Spirillium (e.g. Spirillum minus); Baceteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides uniformis, Bacteroides ureolyticus and Bacteroides vulgatus); Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans); Porphyromonas (e.g. Porphyromonas asaccharolytica, Porphyromonas cangingivalis, Porphyromonas canons, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae); Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium); Chlamydia (e.g. Chlamydia trachomatis); Cryptosporidium (e.g. C. parvum, C. hominis, C. canis, C. fells, C. meleagridis and C. muris); Chlamydophila (e.g. Chlamydophila abortus (Chlamydia psittaci), Chlamydophila pneumoniae (Chlamydia pneumoniae) and Chlamydophila psittaci (Chlamydia psittaci)); Leuconostoc (e.g. Leuconostoc citreum, Leuconostoc cremoris, Leuconostoc dextranicum, Leuconostoc lactis, Leuconostoc mesenteroides and Leuconostoc pseudomesenteroides); Gemella (e.g. Gemella bergeri, Gemella haemolysans, Gemella morbillorum and Gemella sanguinis); and Ureaplasma (e.g. Ureaplasma parvum and Ureaplasma urealyticum).

As used herein, the term “fungi” (and derivatives thereof, such as “fungal infection”) includes, but is not limited to, references to organisms (or infections due to organisms) of the following classes and specific types:

Absidia (e.g. Absidia corymbifera); Ajellomyces (e.g. Ajellomyces capsulatus and Ajellomyces dermatitidis); Arthroderma (e.g. Arthroderma benhamiae, Arthroderma fulvum, Arthroderma gypseum, Arthroderma incurvatum, Arthroderma otae and Arthroderma vanbreuseghemii); Aspergillus (e.g. Aspergillus flavus, Aspergillus fumigatus and Aspergillus niger); Blastomyces (e.g. Blastomyces dermatitidis); Candida (e.g. Candida albicans, Candida glabrata, Candida guilliermondii, Candida krusei, Candida parapsilosis, Candida tropicalis and Candida pelliculosa); Cladophialophora (e.g. Cladophialophora carrionii); Coccidioides (e.g. Coccidioides immitis and Coccidioides posadasii); Cryptococcus (e.g. Cryptococcus neoformans); Cunninghamella (e.g. Cunninghamella sp.); Epidermophyton (e.g. Epidermophyton floccosum); Exophiala (e.g. Exophiala dermatitidis); Filobasidiella (e.g. Filobasidiella neoformans); Fonsecaea (e.g. Fonsecaea pedrosoi); Fusarium (e.g. Fusarium solani); Geotrichum (e.g. Geotrichum candidum); Histoplasma (e.g. Histoplasma capsulatum); Hortaea (e.g. Hortaea werneckii); Issatschenkia (e.g. Issatschenkia orientalis); Madurella (e.g. Madurella grisae); Malassezia (e.g. Malassezia furfur, Malassezia globosa, Malassezia obtusa, Malassezia pachydermatis, Malassezia restricta, Malassezia slooffiae and Malassezia sympodialis); Microsporum (e.g. Microsporum canis, Microsporum fulvum and Microsporum gypseum); Microsporidia; Mucor (e.g. Mucor circinelloides); Nectria (e.g. Nectria haematococca); Paecilomyces (e.g. Paecilomyces variotii); Paracoccidioides (e.g. Paracoccidioides brasiliensis); Penicillium (e.g. Penicillium marneffei); Pichia (e.g. Pichia anomala and Pichia guilliermondii); Pneumocystis (e.g. Pneumocystis jiroveci (Pneumocystis carinii)); Pseudallescheria (e.g. Pseudallescheria boydii); Rhizopus (e.g. Rhizopus oryzae); Rhodotorula (e.g. Rhodotorula rubra); Scedosporium (e.g. Scedosporium apiospermum); Schizophyllum (e.g. Schizophyllum commune); Sporothrix (e.g. Sporothrix schenckii); Trichophyton (e.g. Trichophyton mentagrophytes, Trichophyton rubrum, Trichophyton verrucosum and Trichophyton violaceum); and Trichosporon (e.g. Trichosporon asahii, Trichosporon cutaneum, Trichosporon inkin and Trichosporon mucoides).

Particular bacteria that may be killed using a combination of the invention are Gram-negative bacteria. For example, Enterobacteriaceae, such as Escherichia coli and Enterobacter, Klebsiella (e.g. Klebs. pneumoniae and Klebs. oxytoca), Proteus (e.g. Pr. mirabilis, Pr. rettgeri and Pr. vulgaris); Acinetobacter, and Pseudomonas aeruginosa.

Preferably, the bacteria that may be treated using a combination of the invention include Enterobacteriaceae, such as Escherichia coli and Enterobacter, and Klebsiella, such as Klebs. pneumoniae. More preferably the bacteria are Escherichia coli.

The combinations of the present invention may be used to treat infections associated with any bacterial or fungal organisms, such as those mentioned above; in particular, they may be used for killing multiplying, non-multiplying and/or clinically latent microorganisms associated with such an infection.

Particular conditions which may be treated using the combinations of the present invention include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacillary dysentery, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empyema, endocarditis, endometritis, enteric fever, enteritis, epididymitis, epiglottitis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicameia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis; or infections with MSSA, MRSA, Staph. epidermidis, Strept. agalactiae, Strept. pyogenes, Escherichia coli, Enterobacter, Acinetobacter, Pseudomonas aeruginosa, Klebs. pneumoniae, Klebs. oxytoca, Pr. mirabilis, Pr. rettgeri, Pr. vulgaris, Haemophilis influenzae, Enterococcus faecalis and Enterococcus faecium.

Preferred conditions which may be treated using the combinations of the present invention include those conditions listed above which are caused by Gram-negative bacteria. For example, abscesses, asthma, bacillary dysentery, bacterial conjunctivitis, bacterial keratitis, bacterial vaginosis, bone and joint infections, bronchitis (acute or chronic), brucellosis, burn wounds, cat scratch fever, cellulitis, chancroid, cholangitis, cholecystitis, cystic fibrosis, cystitis, diffuse panbronchiolitis, dental caries, diseases of the upper respiratory tract, eczema, empyema, endocarditis, endometritis, enteric fever, enteritis, epididymitis, epiglottitis, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leptospirosis, liver abscesses, Lyme disease, lymphogranuloma venerium, mastitis, mastoiditis, meningitis and infections of the nervous system, non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, pleural effusion, pneumonia, postoperative wound infections, postoperative gas gangrene, prostatitis, pseudo-membranous colitis, pulmonary emphysema, pyelonephritis, salmonellosis, salpingitis, septic arthritis, septic infections, septicameia, sinusitis, skin infections (e.g. skin granulomas, impetigo, folliculitis and furunculosis), systemic infections, tonsillitis, toxic shock syndrome, tularaemia, typhoid, urethritis, wound infections, yaws, Escherichia coli, Enterobacter, Acinetobacter, Pseudomonas aeruginosa, Klebs. pneumoniae, Klebs. oxytoca, Pr. mirabilis, Pr. rettgeri, Pr. vulgaris or Haemophilis influenzae.

References herein to “treatment” extend to prophylaxis as well as the treatment of established diseases or symptoms.

The combination of the present invention includes a compound of formula (I) or a pharmaceutically acceptable derivative or prodrug thereof. The compound of formula (I) has the following chemical structure:

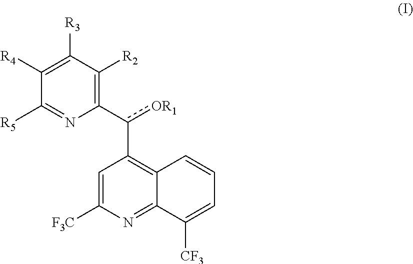

wherein R₁ is H, alkyl, alkenyl or COR^{a}, wherein R^{a} is hydrogen, alkyl, alkenyl, alkynyl, aryl, heteroaryl, carbocyclyl, heterocyclyl, or alkoxy; or R₁ is absent and a double bond is present;
wherein R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl, aryl, alkenyl, cycloalkyl, cycloalkenyl, heteroaryl, carbocyclyl, heterocyclyl, or alkoxy;
or R₂ and R₃, R₃ and R₄ or R₄ and R₅ may together define a cycloalkyl, cycloalkenyl, aryl, heteroaryl or heterocyclyl group;
wherein R₂, R₃, R₄ and R₅ may be the same or different; and
wherein the alkyl, aryl, alkenyl, alkynyl, cycloalkyl, cycloalkenyl, heteroaryl, carbocyclyl, and heterocyclyl groups are optionally substituted.

Representative COR^{a} groups include, but are not limited to, formyl (e.g. —CHO), acetyl, (e.g. —C(O)CH₃), cyclohexylcarbonyl, cyclohexylmethylcarbonyl, benzoyl (e.g. C(O)Ph), benzylcarbonyl (e.g. C(O)CH₂Ph), C(O)—C₁₋₈alkyl, C(O)(CH₂)ₜ(C₈-C₁₀aryl), C(O)(CH₂)ₜ(5-10 membered heteroaryl), C(O)(CH₂)ₜ(C₃-C₁₀cycloalkyl), and C(O)(CH₂)ₜ(4-10 membered heterocycyl), wherein t is an integer from 0 to 4.

R^{a} is preferably selected from C₁-C₈ alkyl or C₁-C₄ alkyl, each optionally substituted with halo, OH, OR^{b}, or NHSO₂R^{b} wherein R^{b} is C₁₋₄ alkyl; or C₃-C₁₀cycloalkyl, 4-10 membered heterocyclyl, C₆-C₁₀aryl, arylalkyl, 5-10 membered heteroaryl or heteroarylalkyl, each optionally substituted with C₁₋₄ alkyl, C₁₋₄ hydroxyalkyl, C₁₋₄ haloalkoxy, OH, OR^{b}, or NHSO₂R^{b} wherein R^{b} is C₁₋₄ alkyl.

Preferably R¹ is H, COR^{a} or absent where R^{a} is defined hereinabove.

COR^{a} may for instance be represented by the formula:

COCR^{c}R^{d}OC(O)R^{e}

wherein R^{c} and R^{d} are each independently H, alkyl, alkenyl, alkynyl, or alkoxy; and R^{e} is alkyl, alkenyl, alkynyl, aryl, heteroaryl, carbocyclyl, heterocyclyl, or alkoxy.
R^{c} is preferably H or a group selected from C₁-C₈ alkyl or C₁-C₄ alkyl, each optionally substituted with halo, OH, OR^{b}, or NHSO₂R^{b} wherein R^{b} is C₁₋₄ alkyl, C₃-C₁₀cycloalkyl, 4-10 membered heterocyclyl, C₆-C₁₀aryl, 5-10 membered heteroaryl, wherein the C₃-C₁₀cycloalkyl, 4-10 membered heterocyclyl, C₆-C₁₀aryl, and 5-10 membered heteroaryl are each optionally substituted with C₁₋₄ alkyl, C₁₋₄ hydroxyalkyl, C₁₋₄ haloalkoxy, OH, OR^{b}, or NHSO₂R^{b} wherein R^{b} is C₁₋₄ alkyl.

R^{d} is preferably a group selected from C₁-C₈ alkyl or C₁-C₄ alkyl, each optionally substituted with halo, OH, OR^{b}, or NHSO₂R^{b} wherein R^{b} is C₁₋₄ alkyl, C₃-C₁₀cycloalkyl, 4-10 membered heterocyclyl, C₆-C₁₀aryl, 5-10 membered heteroaryl, wherein the C₃-C₁₀cycloalkyl, 4-10 membered heterocyclyl, C₆-C₁₀aryl, and 5-10 membered heteroaryl are each optionally substituted with C₁₋₄ alkyl, C₁₋₄ hydroxyalkyl, C₁₋₄ haloalkoxy, OH, OR^{b}, or NHSO₂R^{b} wherein R^{b} is C₁₋₄ alkyl.

Representative COR^{e} groups include, but are not limited to, formyl (e.g. —CHO), acetyl, (e.g. —C(O)CH₃), cyclohexylcarbonyl, cyclohexylmethylcarbonyl, benzoyl (e.g. C(O)Ph), benzylcarbonyl (e.g. C(O)CH₂Ph), C(O)—C₁₋₈alkyl, C(O)(CH₂)ₜ(C₆-C₁₀aryl), C(O)(CH₂)ₜ(5-10 membered heteroaryl), C(O)(CH₂)ₜ(C₃-C₁₀cycloalkyl), and C(O)(CH₂)ₜ(4-10 membered heterocycyl), wherein t is an integer from 0 to 4.

Preferably R^{e} is a group selected from C₁-C₈ alkyl or C₁-C₄ alkyl, each optionally substituted with halo, OH, OR^{b}, or NHSO₂R^{b} wherein R^{b} is C₁₋₄ alkyl, C₃-C₁₀cycloalkyl, 4-10 membered heterocyclyl, C₆-C₁₀aryl, 5-10 membered heteroaryl, wherein the C₃-C₁₀cycloalkyl, 4-10 membered heterocyclyl, C₆-C₁₀aryl, and 5-10 membered heteroaryl are each optionally substituted with C₁₋₄ alkyl, C₁₋₄ hydroxyalkyl, C₁₋₄ haloalkoxy, OH, OR^{b}, or NHSO₂R^{b} wherein R^{b} is C₁₋₄ alkyl; or C₃-C₁₀cycloalkyl, 4-10 membered heterocyclyl, C₆-C₁₀aryl, arylalkyl, 5-10 membered heteroaryl or heteroarylalkyl, each optionally substituted with C₁₋₄ alkyl, C₁₋₄ hydroxyalkyl, C₁₋₄ haloalkoxy, OH, OR^{b}, or NHSO₂R^{b} wherein R^{b} is C₁₋₄ alkyl.

More preferably R^{c} is H or a group selected from C₁-C₈ alkyl or C₁-C₄ alkyl, each optionally substituted with halo, OH, OR^{b}, or NHSO₂R^{b} wherein R^{b} is C₁₋₄ alkyl. Most preferably R^{c} is H or an unsubstituted C₁₋₄ alkyl; and R^{d} is selected from C₁-C₈ alkyl or C₁-C₄ alkyl, each optionally substituted with halo, OH, OR^{b}, or NHSO₂R^{b} wherein R^{b} is C₁₋₄ alkyl.

More preferably R^{d} and R^{e} are independently an unsubstituted C₁₋₄ alkyl group.

Alternatively R¹ may be a group of formula (2) linked via the bond indicated, wherein R^{f} is an optionally substituted C₁₋₄ alkyl group, phenyl or methoxyphenyl. Preferably R^{f} is an unsubstituted C₁₋₄ alkyl, phenyl or methoxyphenyl

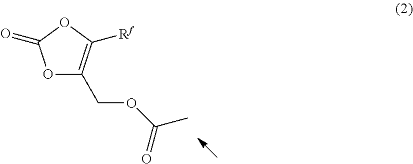

Preferably R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl or alkoxy; or R₂ and R₃, R₃ and R₄ or R₄ and R₅ may together define a cycloalkyl group.

More preferably R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl or alkoxy. For example, R₂, R₃, R₄ and R₅ may each independently be hydrogen, hydroxy, halo, haloalkyl, haloalkoxy, alkyl or alkoxy.

Most preferably R₂, R₃, R₄ and R₅ are each hydrogen.

In one particularly preferred embodiment R₂, R₃, R₄ and R₅ are hydrogen and R₁ is hydrogen or absent. Most preferably R₂, R₃, R₄ and R₅ are hydrogen and R₁ is hydrogen.

Alternatively the combination of the present invention includes a compound of formula (II) or a pharmaceutically acceptable derivative or prodrug thereof. The compound of formula (II) has the following chemical structure:

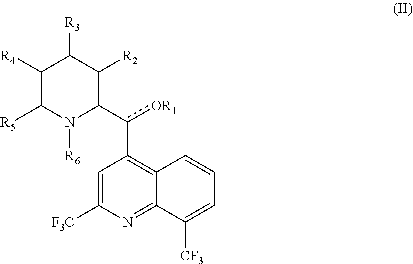

wherein R₁, R₂, R₃, R₄ and R₅ are as defined above for formula (I); and
wherein R₆ is selected from hydrogen, alkyl, aryl, alkenyl, alkynyl, cycloalkyl, cycloalkenyl, heteroaryl, carbocyclyl, or heterocycyl, wherein the alkyl, aryl, alkenyl, alkynyl, cycloalkyl, cycloalkenyl, heteroaryl, carbocyclyl, and heterocyclyl groups are optionally substituted.

Preferably R₆ is hydrogen or an alkyl group. More preferably R₆ is hydrogen.

As used herein, the term “alkyl” includes both saturated straight chain and branched alkyl groups which may be substituted (mono- or poly-) or unsubstituted. Preferably, the alkyl group is a C₁₋₁₅ alkyl group, more preferably a C₁₋₁₀ alkyl group, more preferably still a C₁₋₈ alkyl group, and more preferably still a C₁₋₆ alkyl group. Particularly preferred alkyl groups include, for example, methyl, ethyl, n-propyl, iso-propyl, n-butyl, sec-butyl, tert-butyl, n-pentyl, tert-pentyl, neo-pentyl, iso-pentyl, sec-pentyl and 4-pentyl. In certain embodiments the alkyl group is substituted with halo, OH, OR^{b}, NHSO₂R^{b} wherein R^{b} is C₁₋₄ alkyl.

The term “halo” refers to fluoro, chloro, bromo or iodo.

As used herein, the term “aryl” refers to a C₆₋₁₈ aromatic group which may be substituted (mono- or poly-) or unsubstituted. Preferably the aryl group is a C₆₋₁₄ aryl group, more preferably a C₆₋₁₀ aryl group. Typical examples include phenyl, naphthyl, mesityl, benzyl, and anthracenyl, and a particularly preferred aryl group is phenyl, mesityl or benzyl, e.g. phenyl.

As used herein, the term “alkenyl” refers to a carbon chain containing one or more carbon-carbon double bonds, which may be branched or unbranched, and substituted (mono- or poly-) or unsubstituted. Preferably the alkenyl group is a C₂₋₂₀ alkenyl group, more preferably a C₂₋₁₅ alkenyl group, more preferably still a C₂₋₁₀ alkenyl group, more preferably still a Cₘ alkenyl group, or more preferably still a C₂₋₆ alkenyl group.

As used herein, the term “alkynyl” refers to a carbon chain containing one or more carbon-carbon triple bonds, which may be branched or unbranched, and substituted (mono- or poly-) or unsubstituted. Preferably the alkynyl group is a C₂₋₂₀ alkynyl group, more preferably a C₂₋₁₅ alkynyl group, more preferably still a C₂₋₁₀ alkynyl group, more preferably still a Cₘ alkynyl group, or more preferably still a C₂₋₆ alkynyl group.

As used herein, the term “cycloalkyl” refers to a mono- or multi-ringed cyclic alkyl group which may be substituted (mono- or poly-) or unsubstituted. Preferably the cycloalkyl is a mono-ringed group. Preferably a C₃-C₇ cycloalkyl group, particularly preferred are cyclopentane, cyclohexane and cycloheptane groups, e.g. cyclopentane or cyclohexane. In another embodiment, the cycloalkyl is a multi-ringed group, e.g. adamantyl.

As used herein, the term “heterocyclyl” refers to heteroaryl, heterocycloalkyl and heterocycloalkenyl groups. The term “heteroaryl” refers to an aryl group as defined above wherein at least one ring atom is a heteroatom. Suitable heteroatoms will be apparent to those skilled in the art and include, for example, sulphur, nitrogen, oxygen, phosphorus and silicon. Particularly preferred is when the heteroatom is sulphur, nitrogen or oxygen.

Monocyclic heteroaryl groups include for example, furan, pyrrole, thiophene, imidazole, oxazole, thiazole, 1,3,4-thiadiazole, isothiazole, isoxazole, oxadiazole, oxazole, 1,2,3-oxadiazole, pyrazole, triazole, tetrazole, pyridine, pyrazine, pyrimidine, pyridazines, triazine and tetrazine. Bicyclic or polycyclic heteroaryl groups may include a monocyclic heteroaryl group as defined herein, fused to one or more groups independently selected from an aryl group, a cycloalkyl group, a cycloalkenyl group and another monocyclic heteroaryl group. For example, the heteroaryl group may be indole, benzimidazole, benzothiazole, benzofuran, indoline, quinolone, isoquinoline, isoindole, indazole, phenylpiperidine or benzothiene.

The terms “heterocycloalkyl” and “heterocycloalkenyl” respectively refer to a cycloalkyl group or a cycloalkenyl group as defined above, wherein at least one ring atom in the cycloalkyl or cycloalkenyl group is a heteroatom. Again, suitable heteroatoms will be apparent to those skilled in the art and include, for example, sulphur, nitrogen, oxygen, phosphorus and silicon. Particularly preferred is when the heteroatom is sulphur, nitrogen or oxygen, e.g. aziridine, tetrahydrofuran, pyrrolidine, pyrroline, piperidine, piperazine, thiazolidine, oxazolidine, morpholine, thiane, thiazine, pyrazolidine, pyrazoline, imidazolidine or imidazoline.

The term “alkoxy” refers to an O-alkyl group, wherein alkyl is as defined above. Preferably, the alkoxy group is a C₁₋₂₀ alkoxy group, more preferably a C₁₋₁₅ alkoxy group, more preferably still a C₁₋₁₀ alkoxy group, more preferably still a C₁₋₈ alkoxy group, and more preferably still a C₁₋₈ alkoxy group. Particularly preferred alkoxy groups include, for example, methoxy, ethoxy, iso-propoxy, propoxy, butoxy, iso-butoxy, pentoxy and hexyloxy.

Each of the alkyl, aryl, alkenyl, alkynyl, cycloalkyl, cycloalkenyl, heteroaryl, heterocycloalkyl and heterocycloalkenyl groups described herein may optionally be substituted by one or more substituents selected from alkyl, aryl, alkenyl, alkynyl, cycloalkyl, cycloalkenyl, heteroaryl, heterocycloalkyl, heterocycloalkenyl, halogen, nitro, cyano, silyl, sulfanyl, phosphanyl, hydroxy, alkoxy, amino, CF₃, amide, aminoalkyl, thiol, haloalkyl and haloalkoxy.

Preferably the one or more substituents are selected from, alkyl, halogen, nitro, cyano, hydroxy, alkoxy and amino. More preferably the one or more substituents are selected from C₁₋₈ alkyl, chlorine, bromine, nitro, cyano, hydroxy, C₁₋₈-alkoxy, NH₂, NHC₁₋₄-alkyl, and N(C₁₋₄-alkyl)₂. For example, methyl (Me), ethyl (Et), isopropyl (iPr), chlorine, nitro, hydroxy, MeO, EtO, iPrO, NH₂, NHMe, NHEt, NMe₂ and NEt₂.

Compounds of formula (I) and compounds of formula (II) can be prepared by known methods by those skilled in the art. (2,8-bis-trifluoromethyl-quinolin-4-yl)-pyridin-2-yl-methanone (a compound of formula (I); structure shown above) and (2,8-bis-trifluoromethyl-quinolin-4-yl)-pyridin-2-yl-methanol (a compound of formula (I); structure shown above) are also commercially available from e.g. Sigma Aldrich. HT0160009 (compound of formula (II); structure shown above) is commercially available.

Compounds of formula (I) and compounds of formula (II) may also be chiral molecules with at least one asymmetric carbon centre. In all aspects of the invention, the reference to a compound of formula (I) or a compound of formula (II) thus includes all enantiomers, stereoisomers or diastereoisomers thereof. The corresponding enantiomers and/or stereoisomers and/or diastereoisomers may be isolated or prepared by methods known in the art.

In one embodiment the compound of formula (I) is a racemic mixture of the available enantiomers.

Compounds of formula (I) may also be present as different tautomers. In all aspects of the invention, the reference to a compound of formula (I) thus includes all tautomers thereof

As used herein the term “pharmaceutically acceptable derivative” for the compound of formula (I) and the compound of formula (II) means:

(a) pharmaceutically acceptable salts; and/or
(b) solvates (including hydrates).

Pharmaceutically acceptable salts and solvates (including hydrates) are also understood to include polymorphs such as pseudopolymorphs, packing polymorphs and conformational polymorphs. A review of suitable pharmaceutical salts may be found in Berge et al, J Pharm Sci, 66, 119 (1977) as well as P. H. Stahl and C. G. Wermuth, editors, Handbook of Pharmaceutical Salts: Properties, Selection and Use, Weinheim/Zürich:Wiley-VCH/VHCA, 2002. These texts are incorporated herein by reference.

Suitable acid addition salts include carbon/late salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxybenzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulfonate salts (e.g. benzenesulfonate, methyl-, bromo- or chloro-benzenesulfonate, xylenesulfonate, methanesulfonate, ethanesulfonate, propanesulfonate, hydroxyethanesulfonate, 1- or 2-naphthalene-sulfonate or 1,5-naphthalenedisulfonate salts) or sulfate, pyrosulfate, bisulfate, sulfite, bisulfite, phosphate, monohydrogenphosphate, dihydrogenphosphate, metaphosphate, pyrophosphate or nitrate salts, and the like.

A preferred salt of the compound of formula (I) or the compound of formula (II) is the hydrochloride salt.

The polymyxin is colistin (polymyxin E) or polymyxin B or a pharmaceutically acceptable derivative thereof. By the term “pharmaceutically acceptable derivative” for the polymyxin is meant any known forms of the polymyxin. Such forms are known in the art and include colistin sulfate, colistimethate sodium, and polymyxin B sulfate. Colistimethate sodium is also known as colistin methanesulfonate sodium and colistin sulfomethate sodium.

Particularly preferred for the combination of the present invention is colistin, colistin sulfate or colistimethate sodium.

The polymyxin suitable for use in the combination of the present invention is commercially available, for example from Sigma Aldrich Limited or Finetech Industry Limited.

The invention further includes the compound of formula (I) or the compound of formula (II) in prodrug form, i.e. in the form of a covalently bonded compound which releases the active in vivo. Prodrugs are generally the active ingredient, wherein one or more appropriate groups (typically the OH group) have been modified such that the modification may be reversed upon administration to a human or mammalian subject. Reversion is usually performed by an enzyme naturally present in such subject, though it is possible for a second agent to be administered together with such a prodrug in order to perform the reversion in vivo. Examples of such modifications to the compound of formula (I) or the compound of formula (II) include esters. With an ester prodrug the reversion to the compound may be carried out by an esterase.

Esters are typically formed using organic acids. Organic acids that may be used include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; or with amino acids, for example aspartic or glutamic acid; with benzoic acid. In some cases it may be desirable to prepare double ester type prodrugs such as (acyloxy)alkyl esters or ((alkoxycarbonyl)oxy)alkylesters). Preparations of such ester prodrugs are known in the art. Suitable methods are disclosed in for example J. Med. Chem 1996, 39, 480. These methods are incorporated herein by reference.

Other prodrug systems will be well known to those skilled in the art.

The active ingredients in the combination of the invention may be administered as the raw material but the active ingredients are preferably provided in the form of pharmaceutical compositions.

The active ingredients may be used either as separate formulations or as a single combined formulation. When combined in the same formulation it will be appreciated that the compounds must be stable and compatible with each other and the other components of the formulation.

Formulations of the invention include those suitable for parenteral (including subcutaneous e.g. by injection or by depot tablet, intradermal, intrathecal, intramuscular e.g. by depot and intravenous) and topical (including dermal, buccal and sublingual) or in a form suitable for administration by inhalation or insufflation administration. The most suitable route of administration may depend upon the condition and disorder of the patient. Preferably, the combinations of the invention are formulated for topical, intravenous or inhaled/insufflation administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

When formulated with excipients, the active ingredients may be present in a concentration from 0.1 to 99.5% (such as from 0.5 to 95%) by weight of the total mixture; conveniently from 30 to 95% for tablets and capsules and 0.01 to 50% for liquid preparations.

Topical compositions, which are useful for treating disorders of the skin or of membranes accessible by digitation (such as membrane of the mouth, vagina, cervix, anus and rectum), include creams, ointments, lotions, sprays, gels and sterile aqueous solutions or suspensions. As such, topical compositions include those in which the active ingredients are dissolved or dispersed in a dermatological vehicle known in the art (e.g. aqueous or non-aqueous gels, ointments, water-in-oil or oil-in-water emulsions). Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as Miglyol™, or silicone oils such as dimethicone). Depending, inter alia, upon the nature of the formulation as well as its intended use and site of application, the dermatological vehicle employed may contain one or more components selected from the following list: a solubilising agent or solvent (e.g. a β-cyclodextrin, such as hydroxypropyl β-cyclodextrin, or an alcohol or polyol such as ethanol, propylene glycol or glycerol); a thickening agent (e.g. hydroxymethyl cellulose, hydroxypropyl cellulose, carboxymethyl cellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (e.g. a mixture of dihydrogen phosphate and hydrogen phosphate salts, or a mixture of citric acid and a hydrogen phosphate salt). Topical formulations may also be formulated as a transdermal patch.

Methods of producing topical pharmaceutical compositions such as creams, ointments, lotions, sprays and sterile aqueous solutions or suspensions are well known in the art. Suitable methods of preparing topical pharmaceutical compositions are described, e.g. in WO9510999, U.S. Pat. No. 6,974,585, WO2006048747 and documents cited therein.

Topical pharmaceutical compositions according to the present invention may be used to treat a variety of skin or membrane disorders, such as infections of the skin or membranes (e.g. infections of nasal membranes, axilla, groin, perineum, rectum, dermatitic skin, skin ulcers, and sites of insertion of medical equipment such as i.v. needles, catheters and tracheostomy or feeding tubes) with any of the bacteria, fungi described above, particularly Enterobacteriaceae, such as Escherichia coli and Klebsiella, such as Klebs. pneumoniae.

Topical compositions of the invention may be used for pre-operative surgical hand disinfection, antiseptic hand washing, and pre- and post-operative antisepsis for patients undergoing elective surgery.

Compositions for use according to the invention may be presented in a pack or dispenser device which may contain one or more unit dosage forms containing the active ingredients. The pack may, e.g. comprise metal or plastic foil, such as a blister pack. Where the compositions are intended for administration as two separate compositions these may be presented in the form of a twin pack.

Pharmaceutical compositions may also be prescribed to the patient in “patient packs” containing the whole course of treatment in a single package, usually a blister pack. Patient packs have an advantage over traditional prescriptions, where a pharmacist divides a patients' supply of a pharmaceutical from a bulk supply, in that the patient always has access to the package insert contained in the patient pack, normally missing in traditional prescriptions. The inclusion of the package insert has been shown to improve patient compliance with the physician's instructions.

The administration of the combinations of the invention by means of a single patient pack, or patient packs of each composition, including a package insert directing the patient to the correct use of the invention is a further feature of this invention.

According to a further embodiment of the present invention there is provided a patient pack comprising at least one active ingredient of the combinations according to the invention, i.e. at least one of the compound of formula (I) or a pharmaceutically acceptable derivative or prodrug thereof, or the compound of formula (II) or a pharmaceutically acceptable derivative or prodrug thereof, and a polymyxin selected from polymyxin E and polymyxin B, or a pharmaceutically acceptable derivative thereof, and an information insert containing directions on the use of the combination.

The amount of active ingredients required for use in treatment will vary with the nature of the condition being treated and the age and condition of the patient, and will ultimately be at the discretion of the attendant physician or veterinarian. In general however, doses employed for adult human treatment will typically be in the range of 0.02 to 5000 mg per day, preferably 1 to 1500 mg per day. The desired dose may conveniently be presented in a single dose or as divided doses administered at appropriate intervals, e.g. as two, three, four or more sub-doses per day.

### Biological Tests

Test procedures that may be employed to determine the biological (e.g. bactericidal or antimicrobial) activity of the active ingredients include those known to persons skilled in the art for determining:
(a) bactericidal activity against clinically latent bacteria; and
(b) antimicrobial activity against log phase bacteria.

In relation to (a) above, methods for determining activity against clinically latent bacteria include a determination, under conditions known to those skilled in the art (such as those described in Nature Reviews, Drug Discovery 1, 895-910 (2002), the disclosures of which are hereby incorporated by reference), of Minimum Stationary-cidal Concentration (“MSC”) or Minimum Dormicidal Concentration (“MDC”) for a test compound.

By way of example, WO2000028074 describes a suitable method of screening compounds to determine their ability to kill clinically latent microorganisms. A typical method may include the following steps:
(1) growing a bacterial culture to stationary phase;
(2) treating the stationary phase culture with one or more antimicrobial agents at a concentration and or time sufficient to kill growing bacteria, thereby selecting a phenotypically resistant sub-population;
(3) incubating a sample of the phenotypically resistant subpopulation with one or more test compounds or agents; and
(4) assessing any antimicrobial effects against the phenotypically resistant subpopulation.

According to this method, the phenotypically resistant sub-population may be seen as representative of clinically latent bacteria which remain metabolically active in vivo and which can result in relapse or onset of disease.

In relation to (b) above, methods for determining activity against log phase bacteria include a determination, under standard conditions (i.e. conditions known to those skilled in the art, such as those described in WO2005014585, the disclosures of which document are hereby incorporated by reference), of Minimum Inhibitory Concentration (MIC) or Minimum Bactericidal Concentration (MBC) for a test compound. Specific examples of such methods are described below.

All publications mentioned in the above specification are herein incorporated by reference.

Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### Examples

### Example 1: In Vitro Synergy Effect of a Compound of Formula (I) in Combination with a Polymyxin E Derivative Against Log Phase NDM-1 Klebsiella pneumoniae Subsp. Pneumoniae (BAA2472)

The synergistic effect of a compound of formula (I) (HT0160010) in combination with colistimethate sodium (CMS) was tested against log phase NDM-1 Klebsiella pneumoniae subsp. pneumoniae using chequerboard analysis. As explained hereinabove, HT0160010 has the following chemical structure:

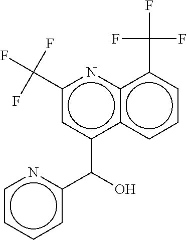

### Materials and Methods

Bacterial strain used: BAA2472 strain of NDM-1 Klebsiella pneumoniae subsp. pneumoniae from ATCC®.
Growth of bacteria: Log phase growth of the bacteria was carried out according to methods known in the art.
Compounds and preparation:
(i) HT0160010 was obtained from a commercial source and dissolved in DMSO to make a stock concentration of 10 mg/ml.
(ii) Colistimethate sodium (CMS) was obtained from a commercial source (e.g. Sigma Aldrich) at a concentration of 10 mg/ml.

Log phase bacterial culture was incubated with HT0160010 and CMS in combination using the chequerboard method known in the art. The overnight culture was diluted with nutrient broth (Oxoid) to 10⁷ CFU/ml and 280 μl of the culture was added to each well to make a final concentration of 300 μl. Incubation of the compounds with the bacterial suspension was carried out for 24 hours. The HT0160010 concentration ranged from 128 to 0 μg/ml and the CMS concentration ranged from 16 to 0 μg/ml.

The effects of the combination were examined by calculating the fractional inhibitory concentration index (FICI) of each combination, as follows: (MIC of drug A, tested in combination)/(MIC of drug A, tested alone)+(MIC of drug B, tested in combination)/(MIC of drug B, tested alone). The interaction of the combination was defined as showing synergy if the FICI was ≤0.5, no interaction if the FICI was ≥0.5 but ≤4.0 and antagonism if the FICI was ≥4.0.

### Results

The chequerboard results are shown below.

| | | CMS | | | | | | | | | | | |
|---|---|---|---|---|---|---|---|---|---|---|---|---|---|
| | | 16 | 8 | 4 | 2 | 1 | 0.5 | 0.25 | 0.125 | 0.0625 | 0.03125 | 0.015625 | 0 |
| HT0160010 | 128 | 0.08 | 0.07 | 0.06 | 0.07 | 0.06 | 0.07 | 0.42 | 0.48 | 0.43 | 0.43 | 0.44 | 0.57 |
| | 64 | 0.06 | 0.05 | 0.05 | 0.05 | 0.05 | 0.05 | 0.33 | 0.35 | 0.36 | 0.36 | 0.40 | 0.56 |
| | 32 | 0.05 | 0.04 | 0.05 | 0.04 | 0.05 | 0.05 | 0.36 | 0.36 | 0.38 | 0.36 | 0.41 | 0.56 |
| | 16 | 0.04 | 0.04 | 0.04 | 0.04 | 0.04 | 0.23 | 0.35 | 0.37 | 0.37 | 0.37 | 0.42 | 0.56 |
| | 8 | 0.04 | 0.04 | 0.04 | 0.04 | 0.04 | 0.32 | 0.35 | 0.41 | 0.36 | 0.38 | 0.41 | 0.57 |
| | 4 | 0.05 | 0.04 | 0.04 | 0.04 | 0.25 | 0.33 | 0.33 | 0.38 | 0.37 | 0.38 | 0.40 | 0.54 |
| | 0 | 0.05 | 0.05 | 0.04 | 0.05 | 0.41 | 0.52 | 0.51 | 0.52 | 0.54 | 0.53 | 0.54 | 0.62 |

### Summary and Conclusions

1. It can be seen from the above chequerboard that there is combination activity between CMS and a compound of formula (I) (HT0160010).
2. The FIC index was calculated as 0.375 showing that there is a significant synergistic effect against NDM-1 K. pneumoniae subsp. pneumoniae when HT0160010 and CMS are used in combination.

### Example 2: In Vitro Synergy Effect of a Compound of Formula (I) in Combination with a Polymyxin E Derivative Against Log Phase NDM-1 Escherichia coli (BAA2469)

The synergistic effect of a compound of formula (I) (HT0160010) in combination with colistimethate sodium (CMS) was tested against log phase NDM-1 E. coli using chequerboard analysis.

### Materials and Methods

Bacterial strain used: BAA2469 strain of NDM-1 E. coli from ATCC®.
Growth of bacteria: Log phase growth of the bacteria was carried out according to methods known in the art.

Compounds and preparation were the same as Example 1. The effects of the combination were also examined by calculating the FICI in the same manner as Example 1.

### Results

| | | CMS | | | | | | | | | | | |
|---|---|---|---|---|---|---|---|---|---|---|---|---|---|
| | | 16 | 8 | 4 | 2 | 1 | 0.5 | 0.25 | 0.125 | 0.0625 | 0.03125 | 0.015625 | 0 |
| HT0160010 | 128 | 0.07 | 0.06 | 0.08 | 0.07 | 0.37 | 0.38 | 0.45 | 0.50 | 0.51 | 0.55 | 0.51 | 0.61 |
| | 64 | 0.06 | 0.05 | 0.06 | 0.05 | 0.29 | 0.35 | 0.39 | 0.51 | 0.52 | 0.56 | 0.53 | 0.64 |
| | 32 | 0.05 | 0.05 | 0.05 | 0.05 | 0.24 | 0.33 | 0.38 | 0.52 | 0.53 | 0.52 | 0.53 | 0.62 |
| | 16 | 0.05 | 0.05 | 0.05 | 0.04 | 0.29 | 0.32 | 0.45 | 0.51 | 0.51 | 0.53 | 0.49 | 0.61 |
| | 8 | 0.05 | 0.05 | 0.04 | 0.07 | 0.30 | 0.36 | 0.48 | 0.49 | 0.49 | 0.52 | 0.53 | 0.58 |
| | 4 | 0.05 | 0.04 | 0.04 | 0.32 | 0.38 | 0.43 | 0.48 | 0.49 | 0.52 | 0.52 | 0.51 | 0.62 |
| | 0 | 0.05 | 0.05 | 0.18 | 0.54 | 0.55 | 0.59 | 0.61 | 0.59 | 0.61 | 0.68 | 0.59 | 0.64 |

### Summary and Conclusions

1. It can be seen from the above chequerboard that there is combination activity between CMS and a compound of formula (I) (HT0160010).
2. The FIC index was calculated as 0.28125 showing that there is a significant synergistic effect against NDM-1 E. coli when HT0160010 and CMS are used in combination.

### Example 3: In Vitro Synergy Effect of a Compound of Formula (II) in Combination with a Polymyxin E Derivative Against Log Phase NDM-1 Klebsiella pneumoniae Subsp. Pneumoniae (BAA2472)

The synergistic effect of a compound of formula (II) (HT0160009) in combination with colistimethate sodium (CMS) was tested against log phase NDM-1 Klebsiella pneumoniae subsp. pneumoniae using chequerboard analysis. As explained hereinabove, HT0160009 has the following chemical structure:

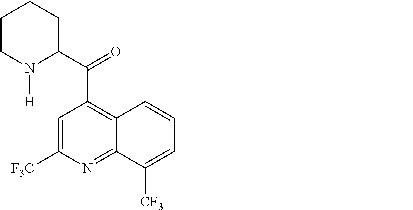 [SMILES: O=C(c1cc(C(F)(F)F)nc2c(C(F)(F)F)cccc12)C1CCCCN1]

### Materials and Methods

Bacterial strain used: BAA2472 strain of NDM-1 Klebsiella pneumoniae subsp. pneumoniae from ATCC®.
Growth of bacteria: Log phase growth of the bacteria was carried out according to methods known in the art.

### Compounds and Preparation:

(i) HT0160009 was obtained from a commercial source and dissolved in DMSO to make a stock concentration of 10 mg/ml.
(ii) Colistimethate sodium (CMS) was obtained from a commercial source (e.g. Sigma Aldrich) at a concentration of 10 mg/ml.

Log phase bacterial culture was incubated with HT0160009 and CMS in combination using the chequerboard method known in the art. The overnight culture was diluted with nutrient broth (Oxoid) to 10⁷ CFU/ml and 280 μl of the culture was added to each well to make a final concentration of 300 μl. Incubation of the compounds with the bacterial suspension was carried out for 24 hours. The HT0160010 concentration ranged from 256 to 0 μg/ml and the CMS concentration ranged from 16 to 0 μg/ml.

The effects of the combination were examined by calculating the FICI in the same manner as Example 1.

### Results

The chequerboard results are shown below.

| | | CMS | | | | | | | | | | | |
|---|---|---|---|---|---|---|---|---|---|---|---|---|---|
| BAA2472 | | 16 | 8 | 4 | 2 | 1 | 0.5 | 0.25 | 0.125 | 0.063 | 0.031 | 0.016 | 0 |
| HT0160009 | 256 | 0.04 | 0.04 | 0.04 | 0.04 | 0.04 | 0.04 | 0.04 | 0.04 | 0.04 | 0.04 | 0.04 | 0.05 |
| | 128 | 0.04 | 0.04 | 0.04 | 0.04 | 0.04 | 0.04 | 0.04 | 0.04 | 0.04 | 0.05 | 0.15 | 0.14 |
| | 64 | 0.04 | 0.04 | 0.04 | 0.04 | 0.04 | 0.04 | 0.04 | 0.15 | 0.16 | 0.17 | 0.18 | 0.17 |
| | 32 | 0.04 | 0.04 | 0.04 | 0.04 | 0.04 | 0.04 | 0.18 | 0.24 | 0.25 | 0.25 | 0.29 | 0.25 |
| | 16 | 0.04 | 0.04 | 0.04 | 0.04 | 0.04 | 0.28 | 0.30 | 0.33 | 0.34 | 0.37 | 0.38 | 0.49 |
| | 8 | 0.04 | 0.04 | 0.04 | 0.04 | 0.04 | 0.34 | 0.34 | 0.37 | 0.37 | 0.40 | 0.38 | 0.50 |
| | 4 | 0.04 | 0.04 | 0.04 | 0.04 | 0.25 | 0.36 | 0.37 | 0.37 | 0.39 | 0.37 | 0.38 | 0.50 |
| | 0 | 0.04 | 0.04 | 0.04 | 0.04 | 0.50 | 0.53 | 0.53 | 0.53 | 0.53 | 0.52 | 0.52 | 0.54 |

### Summary and Conclusions

1. It can be seen from the above chequerboard that there is combination activity between CMS and a compound of formula (II) (HT0160009).
2. The FIC index was calculated as 0.38 showing that there is a synergistic effect against NDM-1 K. pneumoniae subsp. pneumoniae when HT0160009 and CMS are used in combination.

### Example 4: In Vitro Synergy Effect of a Compound of Formula (II) in Combination with a Polymyxin E Derivative Against Log Phase NDM-1 Escherichia coli (BAA2469)

The synergistic effect of a compound of formula (II) (HT0160009) in combination with colistimethate sodium (CMS) was tested against log phase NDM-1 E. coli using chequerboard analysis.

### Materials and Methods

Bacterial strain used: BAA2469 strain of NDM-1 E. coli from ATCC®.
Growth of bacteria: Log phase growth of the bacteria was carried out according to methods known in the art.

Compounds and preparation were the same as Example 3. The effects of the combination were also examined by calculating the FICI in the same manner as Example 3.

### Results

| | | CMS | | | | | | | | | | | |
|---|---|---|---|---|---|---|---|---|---|---|---|---|---|
| BAA2469 | | 16 | 8 | 4 | 2 | 1 | 0.5 | 0.25 | 0.125 | 0.063 | 0.031 | 0.016 | 0 |
| HT0160009 | 256 | 0.05 | 0.05 | 0.05 | 0.05 | 0.05 | 0.05 | 0.05 | 0.05 | 0.05 | 0.05 | 0.05 | 0.05 |
| | 128 | 0.05 | 0.05 | 0.05 | 0.05 | 0.05 | 0.05 | 0.05 | 0.13 | 0.18 | 0.17 | 0.20 | 0.21 |
| | 64 | 0.05 | 0.05 | 0.05 | 0.05 | 0.05 | 0.16 | 0.16 | 0.16 | 0.17 | 0.18 | 0.19 | 0.22 |
| | 32 | 0.05 | 0.05 | 0.05 | 0.05 | 0.06 | 0.20 | 0.20 | 0.21 | 0.25 | 0.26 | 0.27 | 0.32 |
| | 16 | 0.05 | 0.05 | 0.05 | 0.05 | 0.26 | 0.35 | 0.37 | 0.36 | 0.38 | 0.37 | 0.39 | 0.58 |
| | 8 | 0.05 | 0.05 | 0.05 | 0.05 | 0.36 | 0.38 | 0.46 | 0.45 | 0.52 | 0.48 | 0.49 | 0.54 |
| | 4 | 0.05 | 0.05 | 0.05 | 0.28 | 0.40 | 0.49 | 0.52 | 0.55 | 0.54 | 0.52 | 0.52 | 0.61 |
| | 0 | 0.05 | 0.05 | 0.05 | 0.55 | 0.56 | 0.62 | 0.62 | 0.63 | 0.66 | 0.64 | 0.62 | 0.69 |

### Summary and Conclusions

1. It can be seen from the above chequerboard that there is combination activity between CMS and a compound of formula (I) (HT0160010).
2. The FIC index was 0.38 showing that there is a significant synergistic effect against NDM-1 E. coli when HT0160009 and CMS are used in combination.

1. A combination comprising: (A) a compound of Formula (I) or a pharmaceutically acceptable derivative or prodrug thereof and a polymyxin selected from polymyxin E and polymyxin B or a pharmaceutically acceptable derivative thereof; or (B) a compound of Formula (II) or a pharmaceutically acceptable derivative or prodrug thereof and a polymyxin selected from polymyxin E and polymyxin B or a pharmaceutically acceptable derivative thereof, wherein Formula (I) has the following formula: 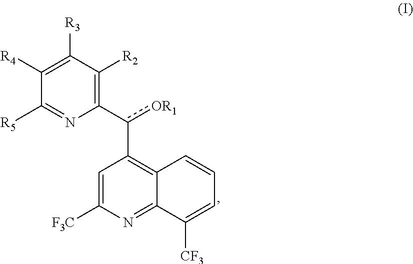
wherein R₁ is hydrogen, alkyl, alkenyl or COR^{a}, wherein R^{a} is hydrogen, alkyl, alkenyl, alkynyl, aryl, heteroaryl, carbocyclyl, heterocyclyl, or alkoxy, or R₁ is absent and a double bond is present,
wherein R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl, aryl, alkenyl, cycloalkyl, cycloalkenyl, heteroaryl, carbocyclyl, heterocyclyl, or alkoxy, or wherein R₂ and R₃, R₃ and R₄ or R₄ and R₅ may together define a cycloalkyl, cycloalkenyl, aryl, heteroaryl or heterocyclyl group, wherein R₂, R₃, R₄ and R₅ may be the same or different, and
wherein the alkyl, aryl, alkenyl, alkynyl, cycloalkyl, cycloalkenyl, heteroaryl, carbocyclyl, and heterocyclyl groups are optionally substituted groups;
wherein Formula (II) has the following formula: 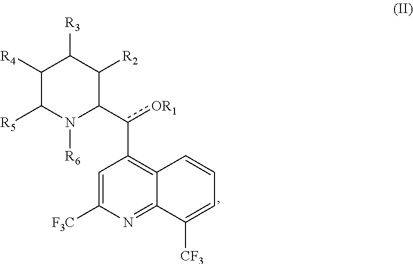
and
wherein R₁, R₂, R₃, R₄ and R₅ are as defined above and wherein R₆ is selected from hydrogen, alkyl, aryl, alkenyl, alkynyl, cycloalkyl, cycloalkenyl, heteroaryl, carbocyclyl, or heterocycyl, wherein the alkyl, aryl, alkenyl, alkynyl, cycloalkyl, cycloalkenyl, heteroaryl, carbocyclyl, and heterocyclyl groups are optionally substituted groups.

2. (canceled)

3. The combination according to claim 1, wherein R₁ is hydrogen.

4. The combination according to claim 1, wherein R₁ is absent.

5. The combination according to claim 1, wherein R₂, R₃, R₄ and R₅ are each independently hydrogen, hydroxy, amino, aminoalkyl, thiol, halo, haloalkyl, haloalkoxy, cyano, nitro, silyl, sulfanyl, phosphanyl, alkyl, alkenyl, or alkoxy.

6. The combination according to claim 1, wherein R₂, R₃, R₄ and R₅ are each independently hydrogen, hydroxy, halo, haloalkyl, haloalkoxy, alkyl, aryl, alkenyl, or alkoxy.

7. The combination according to claim 1, wherein R₂, R₃, R₄ and R₅ are hydrogen.

8. The combination according to claim 1, wherein R₆ is hydrogen or an alkyl group.

9. The combination according to claim 8, wherein R₆ is hydrogen.

10. The combination according to claim 1, wherein the polymyxin is polymyxin E or a pharmaceutically acceptable derivative thereof.

11. The combination according to claim 1 for use in the treatment of a microbial infection.

12. The combination according to claim 11 for use in killing multiplying, non-multiplying or clinically latent microorganisms associated with a microbial infection.

13. The combination of claim 1 for use in the manufacture of a medicament for the treatment of a microbial infection, in particular for killing multiplying, non-multiplying and/or clinically latent microorganisms associated with such an infection.

14. A method of treating a microbial infection, wherein the method comprises administering the combination according to claim 1 to a subject in need thereof.

15. The combination according to claim 11, wherein the microbial infection is a bacterial infection.

16. The combination according to claim 15, wherein the microbial infection is caused by Enterobacteriaceae, Klebsiella, Proteus, Acinetobacter or Pseudomonas aeruginosa.

17. The combination according to claim 16, wherein the microbial infection is caused by Enterobacteriaceae or Klebsiella.

18. The combination according to claim 17, wherein the microbial infection is caused by E. coli from the Enterobacteriaceae family, e.g. E. coli.

19. A pharmaceutical composition comprising: (a) the compound of Formula (I) as defined in claim 1; (b) a polymyxin selected from polymyxin E and polymyxin B or a pharmaceutically acceptable derivative thereof; and (c) a pharmaceutically acceptable adjuvant, diluent or carrier.

20. A pharmaceutical composition comprising: (i) the compound of Formula (II) as defined in claim 1; (ii) a polymyxin selected from polymyxin E and polymyxin B or a pharmaceutically acceptable derivative thereof; and (iii) a pharmaceutically acceptable adjuvant, diluent or carrier.

21. The pharmaceutical composition according to claim 19, wherein the polymyxin is polymyxin E or a pharmaceutically acceptable derivative thereof.

22. The pharmaceutical composition according to claim 19 for use in treating a microbial infection.

23. (canceled)

24. (canceled)
